(19) United States
(12) Patent Application Publication
(10) Pub. No.: US 2024/0350946 A1
(43) Pub. Date: Oct. 24, 2024

(54) METHODS AND SYSTEMS FOR ISOLATING VERY SMALL EMBRYONIC STEM CELLS (VSELs) FROM BLOOD

(71) Applicant: Mark Kyle, Austin, TX (US)

(72) Inventor: Mark Kyle, Austin, TX (US)

(21) Appl. No.: 18/641,917

(22) Filed: Apr. 22, 2024

Related U.S. Application Data
(60) Provisional application No. 63/461,152, filed on Apr. 21, 2023.

(51) Int. Cl. B01D 21/26; A61M 1/36

(52) U.S. Cl. CPC B01D 21/262; A61M 1/3693; A61M 2202/0413; B01D 2221/10

(57) ABSTRACT

Isolating VSELs utilizing a centrifuge, filter, and an ultra-sonification bath, wherein the isolated VSELs are injected into a target area of an animal. Utilizing a centrifuge and ultra-sonification bath may be used to isolate VSELs without damaging the VSELs. Specifically, blood positioned in centrifuge tubes may be spun at specific RCFs, for specific windows at times, within specific temperature ranges, which may allow the VSELs to be harvested at sufficient amounts to be effective.

### BACKGROUND INFORMATION

### Field of the Disclosure

Examples of the present disclosure relate to systems and methods for isolating VSELs from an animal's peripheral blood. Specifically, embodiments are directed towards isolating VSELs utilizing centrifuge cycles, filtering cycles, and an ultra-sonification bath, wherein the isolated VSELs are injected in a target area of an animal.

### Background

In molecular organisms, stem cells are cells that can differentiate into various types of cells and proliferate indefinitely to produce more of the same stem cell. VSELs represent a population of extremely small cells that are negative for lineage markers. Their embryonic-like characteristics include the expression of markers of pluripotency; the ability to give rise to cellular derivatives of all three germ-layers; and the ability to form embryoid-like bodies.

After tissue injury, including acute myocardial infarction (MI), VSELs can be mobilized into the peripheral blood and repair the damaged organ. Given the ability of VSELs to differentiate into cardiomyocytes and endothelial cells, and their ability to secrete various cardioprotective growth factors/cytokines, VSELs may serve as an ideal cellular source for cardiac repair and other alignments.

However, conventional methods to isolate stem cells from an animal require anesthetics, where fat is harvested from the animal's stomach, tissue, etc. or multipotent stem cells from bone marrow This carries significant risks associated with anesthetics, smaller animals generally have insufficient amounts of fat that restricts the size of a sample that can be harvested, and requires a sufficient amount of time and resources.

Furthermore, stem cells harvested from fat or bone marrow are not pluripotent, and conventional methods to isolate the stem cells are not efficient in isolating sufficient amounts of stem cells or damage the stem cells.

Accordingly, needs exist for non-invasive systems and methods for isolating VSELs from peripheral blood, which are readily available and used in clinical settings.

### SUMMARY

Embodiments are directed towards non-invasive systems and methods for isolating VSELs from peripheral blood, which are readily available and used in clinical settings.

In embodiments, a blood sample from a small animal, such as a dog, cat, etc. may be performed. The blood may be collected using needles, syringes, and other equipment. In embodiments, 5 ml to 450 ml of peripheral blood that is autologous may be collected. Next, approximately 15 ml of the collected peripheral blood may be transferred into separate centrifuge tubes of equal amounts.

The separate-first-centrifuge tubes may be inserted into a centrifuge, and spun at 250 relative centrifugal force (RCF) with a cool temperature, such as approximately 5 degrees Celsius, for 30 mins. In embodiments, the cool temperature may be above freezing and below 10 degrees Celsius. The cool temperature may be useful in promoting the activity of the plasma within the red blood cells.

Spinning and cooling the peripheral blood may be utilized to separate the plasma from the red blood cells within the separate centrifuge tubes, wherein the plasma may be positioned on top of the red blood cells within the first centrifuge tubes. In embodiments, a layer of white blood cells may be positioned between the plasma and the red blood cells.

Subsequently, the plasma may be removed from the first centrifuge tube, via a pipette, and positioned in a new-second-centrifuge tube. The plasma within the second centrifuge tube may be run through a filter, such as a 5-micron filter, to remove any remaining white blood cells, larger stem cells, and any residual red blood cells.

The remaining, filtered plasma within the second centrifuge tube may be run at approximately 1300 RCF for approximately 20 minutes. This centrifugation may isolate the stem cell pellets at the bottom of the second centrifuge tube, wherein the plasma is positioned on top of the stem cell pellets within the second centrifuge tube. The plasma may be harvested from the second centrifuge tube and positioned into a third tube, wherein the stem cell pellets may remain at the bottom of the first centrifuge tube. For example, the plasma may be positioned into a 15 ml tube and refrigerated.

The third 15 ml tube may now be considered Platelet Rich Plasma (PRP), and put into an ultra-sonification bath to lysate the platelets for 20 mins. The ultra-sonification bath will break down the membrane of the PRP plasma, releasing the growth factors of the PRP to help activate the VSELs and anti-inflammator within the PRP.

The third 15 ml tube may be removed and put into a centrifuge and spun at 2000-3000 RCFs for 20 mins. This may assist in separating debris from the lysate platelets. The resulting plasma may be now rich in growth factors (PRGF).

The VSELs within the refrigerated second centrifuge tube may be combined with the plasma within the third centrifuge tube to resuspend the stem cells to activate the pluripotent VSELs. Once the VSELs are suspended within the plasma, the VSELs and plasma may be placed through a 5-micron filter, which will result in activated VSELs that are 2.5-3 micrometers in diameter. The filtered, large, and activated VSELs may be transferred into vials to be injected into a targeted area of the animal.

These, and other, aspects of the invention will be better appreciated and understood when considered in conjunction with the following description and the accompanying drawings. The following description, while indicating various embodiments of the invention and numerous specific details thereof, is given by way of illustration and not of limitation. Many substitutions, modifications, additions, or rearrangements may be made within the scope of the invention, and the invention includes all such substitutions, modifications, additions, or rearrangements.

### BRIEF DESCRIPTION OF THE DRAWINGS

Non-limiting and non-exhaustive embodiments of the present invention are described concerning the following figures, wherein reference numerals refer to like parts throughout the various views unless otherwise specified.

FIG. 1 depicts a method for isolating VSELs from peripheral blood, according to an embodiment.

Corresponding reference characters indicate corresponding components throughout the several views of the drawings. Skilled artisans will appreciate that elements in the figures are illustrated for simplicity and clarity and have not necessarily been drawn to scale. For example, the dimensions of some of the elements in the figures may be exaggerated relative to other elements to help improve understanding of various embodiments of the present disclosure. Also, common but well-understood elements that are useful or necessary in a commercially feasible embodiment are often not depicted to facilitate a less obstructed view of these various embodiments of the present disclosure.

### DETAILED DESCRIPTION

In the following description, numerous specific details are outlined to provide a thorough understanding of the present embodiments. It will be apparent, however, to one having ordinary skill in the art, that the specific detail need not be employed to practice the present embodiments. In other instances, well-known materials or methods have not been described in detail to avoid obscuring the present embodiments.

Embodiments are directed toward ways to utilize stem cell therapies that are non-invasive and do not utilize adipose (fat) stem cells or bone marrow stem cells. Conventional systems for stem cell therapies utilize multipotent stem cells that cannot differentiate into all cell types within a particular lineage.

However, embodiments are directed towards utilizing peripheral blood to harvest VSELs that represent a population of extremely small nonhematopoietic pluripotent cells, wherein the pluripotent cells can give rise to all of the cell types that make up the body.

Embodiments may utilize a centrifuge and ultra-sonification bath to isolate VSELs without damaging the VSELs. Specifically, blood positioned in centrifuge tubes may be spun at specific RCFs, for specific windows at times, within specific temperature ranges, which may allow the VSELs to be harvested at sufficient amounts to be effective.

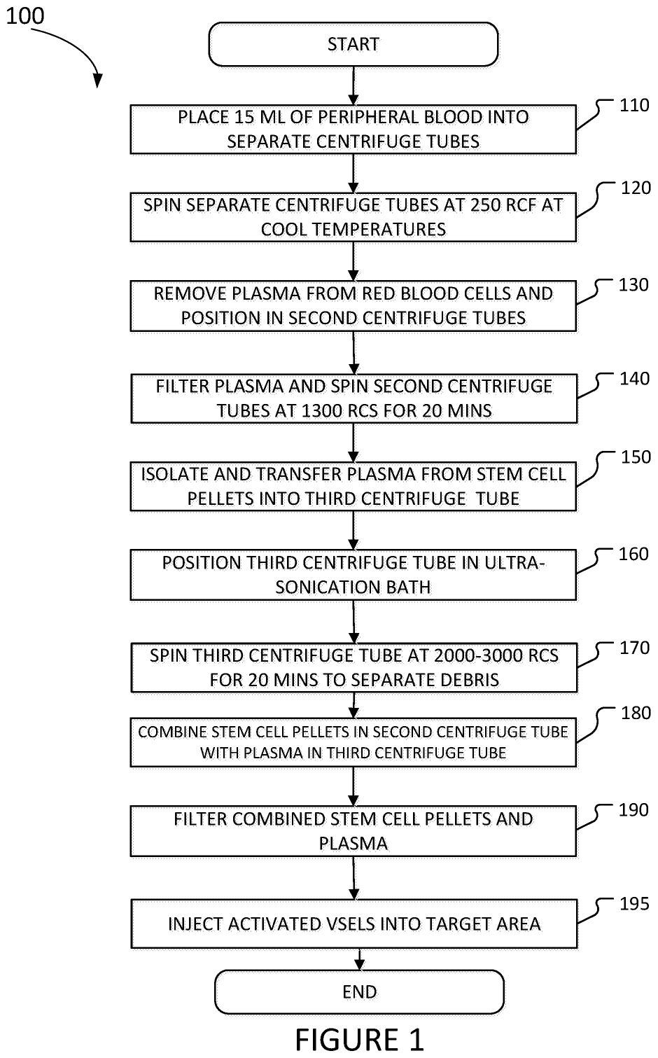

FIG. 1 depicts a method 100 for isolating VSELs from peripheral blood, according to an embodiment. The operations of method 100 presented below are intended to be illustrative. In some embodiments, method 100 may be accomplished with one or more additional operations not described, and/or without one or more of the operations discussed. Additionally, the order in which the operations of method 100 are illustrated in FIG. 1 and described below is not intended to be limiting.

At operation 110, a blood sample from a small animal, such as a dog, cat, etc. may be obtained. The blood may be collected using needles, syringes, and other equipment. In embodiments, 5 ml to 450 ml of peripheral blood that is autologous may be collected. The 15 ml of the collected peripheral blood may be transferred into separate centrifuge tubes of equal amounts.

At operation 120, the separate-first-centrifuge tubes may be inserted into a centrifuge, and spun at 250 relative centrifugal force (RCF) with a cool temperature, such as approximately 5 degrees Celsius, for 30 mins. In embodiments, the cool temperature may be above freezing and below 10 degrees Celsius. The cool temperature may be useful in promoting the activity of the plasma within the red blood cells. Furthermore, spinning and cooling the peripheral blood may be utilized to separate the plasma from the red blood cells within the separate centrifuge tubes, wherein the plasma may be positioned on top of the red blood cells within the first centrifuge tubes. In embodiments, a layer of white blood cells may be positioned between the plasma and the red blood cells.

At operation 130, the plasma may be removed from the first centrifuge tubes, such as via a pipette, and positioned in a new-second-centrifuge tube.

At operation 140, the plasma within the second centrifuge tube may be run through a filter, such as a 5-micron filter, to remove any remaining white blood cells, larger stem cells, and any residual red blood cells.

At operation 150, the remaining, filtered plasma within the second centrifuge tube may be run at approximately 1300 RCF for approximately 20 minutes. This centrifugation may isolate the stem cell pellets at the bottom of the second centrifuge tube, wherein the plasma is positioned on top of the stem cell pellets within the second centrifuge tube. The plasma may be harvested from the second centrifuge tube and positioned into a third tube, wherein the stem cell pellets may remain at the bottom of the first centrifuge tube. For example, the plasma may be positioned into a 15 ml tube and refrigerated.

At operation 160, the third 15 ml tube may now be considered Platelet Rich Plasma (PRP), and put into an ultra-sonification bath to lysate the platelets for 20 mins. The ultra-sonification bath will break down the membrane of the PRP plasma, releasing the growth factors of the PRP to help activate the VSELs and anti-inflammatories within the PRP.

At operation 170, the third 15 ml tube may be removed, and put into a centrifuge spun at 2000-3000 RCFs for 20 mins. This may assist in separating debris from the lysate platelets. The resulting plasma may be now rich in growth factors (PRGF).

At operation 180, the VSELs within the refrigerated second centrifuge tube may be combined with the plasma within the third centrifuge tube to resuspend the stem cells to activate the pluripotent VSELs.

At operation 185, once the VSELs are suspended within the plasma, the VSELs and plasma may be placed through a 5-micron filter, which will result in activated VSELs that are 2.5-3 micrometers in diameter.

At operation 190, the filtered, large, and activated VSELs may be transferred into vials to be injected into a targeted area of the animal.

Reference throughout this specification to “one embodiment”, “an embodiment”, “one example” or “an example” means that a particular feature, structure, or characteristic described in connection with the embodiment or example is included in at least one embodiment of the present invention. Thus, appearances of the phrases “in one embodiment”, “in an embodiment”, “one example” or “an example” in various places throughout this specification are not necessarily all referring to the same embodiment or example. Furthermore, the particular features, structures, or characteristics may be combined in any suitable combinations and/or sub-combinations in one or more embodiments or examples. In addition, it is appreciated that the figures provided herewith are for explanation purposes to persons ordinarily skilled in the art and that the drawings are not necessarily drawn to scale.

Although the present technology has been described in detail for illustration based on what is currently considered to be the most practical and preferred implementations, it is to be understood that such detail is solely for that purpose and that the technology is not limited to the disclosed implementations, but, on the contrary, is intended to cover modifications and equivalent arrangements that are within the spirit and scope of the appended claims. For example, it is to be understood that the present technology contemplates that, to the extent possible, one or more features of any implementation can be combined with one or more features of any other implementation.

What is claimed is:

1. A method for isolation very small embryonic stem cells from peripheral blood of a small animal, the method comprising:
obtain a blood sample from the small animal;
position the blood sample in a first centrifuge tube and a second centrifuge tube;
insert a first centrifuge tube and a second centrifuge tube in a centrifuge, and spin the centrifuge tube at 250 relative centrifugal force at a temperature above freezing and below ten degrees temperature;
remove plasma from the first centrifuge tube and second centrifuge tube, and position the removed plasma in a third centrifuge tube;
run plasma within the third centrifuge tube through a filter to remove white blood cells, larger stem cells, and residual red blood cells from the plasma;
run the remaining plasma in a third centrifuge tube at 1300 relative centrifugal force to isolate stem cell pellets at the bottom of the third centrifuge tube; and
position plasma without the stem cell pellets in a fourth centrifuge tube, and put the fourth centrifuge tube ultra-sonification bath to lysate platelets.
